(19) United States
(12) Patent Application Publication
(10) Pub. No.: US 2011/0122990 A1
(43) Pub. Date: May 26, 2011

(54) METHODS APPARATUS ASSEMBLIES AND SYSTEMS FOR IMPLEMENTING A CT SCANNER

(76) Inventor: Ehud Dafni, Caesarea (IL)

(21) Appl. No.: 12/953,583

(22) Filed: Nov. 24, 2010

Related U.S. Application Data
(60) Provisional application No. 61/281,857, filed on Nov. 24, 2009.

(51) Int. Cl. A61B 6/03

(52) U.S. Cl. 378/4

(57) ABSTRACT

Disclosed is a Computer Tomography (CT) system including a gantry having first and second semicircular support elements which are concentric with one another. Each of the first and second semicircular support elements has a missing sector and at least one of the missing sectors may be of an angle of 180 degrees or less. A controller may be adapted to regulate relative rotational positions between said first and second support elements.

### RELATED APPLICATIONS

The present application claims priority from U.S. Prov. App. No. 61/281,857, filed on Nov. 24, 2009, all of which is hereby incorporated by reference in its entirety.

### FIELD OF THE INVENTION

The present invention relates generally to the field of imaging by X-Ray Computer Tomography (CT). More specifically, the present invention relates to methods, apparatus, assemblies and systems of implementing a CT scanner with a semi-circular (e.g. C shaped) gantry for convenient positioning of the scanned subject in the scan field.

### BACKGROUND

X ray computed tomography (CT) is widely used in medical imaging and other fields. The basic principle behind CT scanning includes characterizing each of a set of volume elements in a volume being scanned by transmitting radiation through each of the volume elements from multiple angles. Each exposure at each angle of a CT scan produces a one or two dimensional image on a detector, where the intensity of exposure on a detector element of the detector array indicates an average attenuation of a transmitted ray caused by matter along a direct path between the radiation source and the detector element. Algorithms known in the art as filtered back projection or other algorithms are used to reconstruct images of the scanned volume out of the attenuation data.

In third generation CT scanners the X-ray source and detector are mounted opposite each other on a rotor and data is acquired while the X-ray source and detector rotate simultaneously about the scanned subject. In fourth generation CT scanners a detector array covering a certain angular sector around the patient is stationary, whereas the X-ray source mounted on a rotor rotates on the opposite side of the scanned subject.

Many CT scanners known in the art comprise a stationary gantry structure and a rotor structure capable of rotating relative to the gantry wherein the scanned subject is positioned in a bore through the gantry. FIG. 1a shows a typical prior art medical scanner. As can be seen in FIG. 1a, the patient is supported on a patient support in a lying position and inserted into the scanner bore by horizontal feed of the patient support. In homeland security applications, for example, scanned subject positioning is achieved by feeding scanned luggage into the scanner bore by a conveyer. While the closed “O” shaped gantry of FIG. 1a provides satisfactory solutions for many applications, it imposes restrictions on subject loading into the scan field. For example, a patient already lying on a surgical table during a surgical procedure cannot be inserted into the scan field unless the table is cantilevered. Application of scanners with this geometry for body scans of patients in sitting or standing positions is cumbersome. Scanning of long industrial objects such as pipes or logs is also limited.

Another construction of CT scanners known in the art is based on “C arm” or “U arm” geometry as shown in FIG. 1b. This approach provides convenient loading into the scan field and access to the patient while in the scan field. But for body scans the C arm frame has to be rather large and bulky since it has to encompass the entire patient upper or lower body, depending on loading direction and lying patient support still has to be cantilevered. For long industrial objects extending out of the scan field this geometry is not practical (except for the ends of the objects). C arms may be rotated also in a plane parallel to the C frame by sliding the C frame relative to the support frame. However, the angular range that may be covered by the source and detector in such motion in prior art C arms is limited to the angular range covered by the C frame.

U.S. Pat. No. 6,940,941 to Gregerson et al., the content of which is incorporated herein by reference, provides a breakable gantry of a CT scanner wherein the rotor forms a partial ring about the patient and the stationary part of the gantry forms a complete “O” shaped frame but it can be broken by removing a section of the gantry for sideway subject loading. The gantry of the U.S. Pat. No. 6,940,941 must be unbroken before scanning takes place.

The purpose of the present invention is to provide an alternative method, apparatus, assembly and system for CT scanning that enables convenient loading of the scanned subject under a variety of conditions while overcoming certain limitations of prior art CT scanners.

### SUMMARY OF THE INVENTION

The present invention includes methods, apparatus, assemblies and systems for implementing computed tomography (CT) imaging. According to some embodiments of the present invention, a CT imaging system may include a gantry constructed from two semi-circular (e.g. “C”-shaped) support elements, a first support element which may be adapted to be stationary (stationary support element), and a second support element which may be adapted to rotate relative to the first element (rotating support element). Each of the semi-circular elements of the assembly may have a semi-circular shape greater than 180° degree (180°). According to further embodiments of the present invention, the rotating element may be supported by, concentric with and rotate in substantially the same plane as the stationary element. According to further embodiments of the present invention, each of the gantry elements may encompass sector having a (rotational) angle greater than 180° and may have a missing sector with a (rotational) angle of less than 180°. The missing sectors of each of the respective support elements may define an opening into and out of the center of the support elements. When the missing sectors of each of the support elements are completely or partially aligned (i.e. when the rotating element is rotated relative to the stationary element such that the missing sectors are aligned) with one another, the openings may form or define an opening into and out of a scanning area of the CT gantry. The scanning area or field of the CT gantry may be substantially at and/or around the center of each of the semi-circular support elements.

According to further embodiments, the rotating support element may support or be otherwise functionally associated with a CT X-ray source, which X-ray source may be mounted upon or otherwise structurally associated with the rotating support element. An X-ray sensor array also be mounted upon or otherwise structurally associated with the rotating support element, optionally at or near a point 180° offset from the X-ray source.

According to some embodiments of the present invention, the gantry may be in a “loading” state (i.e. loading of the subject to be scanned) when the missing sectors of each respective support element are substantially overlapping. The overlapping of the missing portions/sectors of the respective support elements may form an opening suitable for positioning of the subject to be scanned in the scan field.

According to further embodiments of the present invention, during CT scanning of a subject loaded into the gantry, the rotating element may rotate about the subject and the X-ray source may be energized (activated) intermittently, constantly, or substantially constantly.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1a shows a schematic description of an exemplary prior art CT system having an “O” shaped gantry;

FIG. 1b shows a schematic description of an exemplary prior art CT system having a “C” shaped gantry;

FIGS. 2a & 2b is a schematic description of an exemplary system according to some embodiments of the present invention in two different positions, the loading position (2a) and another position (2b);

FIG. 3a is a schematic description according to some embodiments of the present invention showing an example of the rotor supported by support wheels attached to the stator;

FIG. 3b is a schematic description according to some embodiments of the present invention showing an example of the support wheel geometry;

FIG. 4 is a schematic description according to some embodiments of the present invention of an exemplary rotor support system having a carriage which may be attached to the stator or rotor, riding on a track which may be attached to the rotor or stator;

FIG. 5a is a schematic description of an exemplary mechanism using a cogwheel to rotate the rotor, according to some embodiments of the present invention;

FIG. 5b is a schematic description of an exemplary mechanism according to some embodiments of the present invention, using two of the support wheels to rotate the rotor by friction;

FIGS. 6a & 6b is a schematic description of an exemplary fourth generation CT system according to some embodiments of the present invention showing the system in the loading position (6a) and in a scanning position (6b) in which the X-rays radiated from the source attached to the rotor impinge the detector attached to the stator through the missing sector in the rotor; and

FIGS. 7a-7c show several exemplary applications, configurations, installations and orientations of the system according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

Embodiments of the present invention may include a Computer Tomography (CT) system for scanning a subject. The system may include a gantry having a first semicircular stationary support element and a second semicircular rotational support element adapted to rotate concentric to the first support element by at least 360 degrees. Each of the first and second semicircular support elements may have a missing sector and the missing sectors may also be missing during rotation. The system may further include an X ray source mounted on the rotational support element and adapted to irradiate the subject from multiple view angles. The system may further include an X Ray detector array adapted to receive X ray radiation that was emitted by the source and attenuated by said subject.

The system may further comprise control circuitry adapted to regulate: (1) relative rotational positions between said first and second elements, (2) activation of said X ray source, and (3) acquisition of attenuation data and reconstruction of CT images. The control circuitry may comprise a single controller or a multiplicity of controllers. The system may further include an electromechanical actuator functionally associated with the control circuitry, wherein responsive to signaling from said control circuitry the actuator may be adapted to rotate the rotating support element concentrically around a common center of the two concentric support elements.

The system may be adapted such that a subject is loaded to scan position in a lateral direction respective the gantry. During subject loading the control circuitry may cause the electromechanical actuator to rotate said rotating support element into a position such that the missing sectors of both support elements are at least partially overlapping. The system may be adapted such that during subject scanning the control circuitry causes said electromechanical actuator to rotate the rotating support element at least 180 degrees relative to said stationary support element. During scanning, the subject may be scanned during rotation of at least 180 degrees. During scanning, the subject may be scanned during rotation of at least 360 degrees.

The gantry rotation plan may be vertical and the scan subject may be a human in a horizontal laying position. The gantry rotation plan may be horizontal and the scan subject may be a human in a standing or upright sitting position. The gantry may be pivotally mounted such that its central axis may be changes in order to accommodate scan subjects in various orientations.

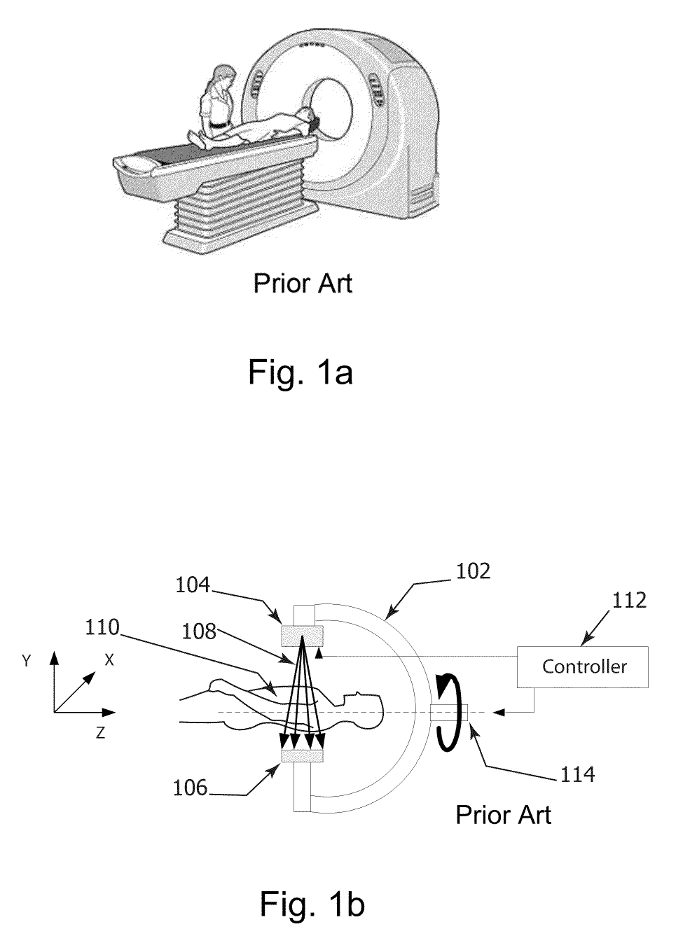

FIG. 1a is an exemplary illustration of a prior art conventional medical CT scanner based on “O” shaped gantry. FIG. 1b is an exemplary illustration of a prior art CT scanner based on “C arm” geometry and some major components of the system. “C” (or “U”) shaped frame 102 may carry X-ray source 104 and detector 106. X-ray beam 108 may impinge on scanned subject 110 and the radiation attenuated by the subject may be detected by detector 106. Controller 112 may be adapted to rotate the frame 102 about axis 114, and may be adapted to activate X-rays source 104, and may also be adapted to acquire projection data and reconstruct the projection data to images. In a coordinate system where the gantry rotation is about the Z axis, and the Y axis points from the detector to the source, the rotation plane is the (X,Y) plane and the C arm frame in this particular example is spanned across the (Y,Z) plane. There are certain prior art X-ray imaging systems based on C arms or other similar designs which may be capable of rotating about the imaged subject wherein the frame is spanned across the rotation plane. However such systems may have a limited rotation range and may not be capable of acquiring 180° of projection data or more as may be desired for CT scanning.

According to some embodiments of the present invention, there may be a CT scanning system which may comprise a gantry, an X-ray source, and a detector. According to some embodiments of the present invention, the CT scanning system may also comprise a collimator. According to some embodiments of the present invention, the CT scanning system may also comprise a controller. According to some embodiments of the present invention, the CT scanning system may also comprise other elements and modules which are standard in CT scanning systems, such as an image reconstruction module, a display, a control console, and image storage. According to some embodiments of the present invention, the CT scanning system gantry may be constructed from two elements, a first gantry element and a second gantry element. According to some embodiments of the present invention, the first gantry element may be stationary (referred to hereinafter as “Stator”), and the second gantry element may be able to rotate relative to the stator (referred to hereinafter as “Rotor”). According to some embodiments of the present invention, the inner side of the stator may have a shape of the letter “C”. According to some embodiments of the present invention, the open sector of the C-shaped stator may be less than 180°. According to some embodiments of the present invention, the inner side of the C-shaped stator may be circular. According to some embodiments of the present invention, the rotor may have a shape of the letter “C”. According to some embodiments of the present invention, the open sector of the C-shaped rotor may be less than 180°. According to some embodiments of the present invention, the C-shaped rotor may be a circular structure segment. According to some embodiments of the present invention, the rotor's rotation axis may be at the center of the inner side of the stator. According to some embodiments of the present invention, the rotor may be rotated by an actuator. According to some embodiments of the present invention, the rotor may be centered within the stator. According to some embodiments of the present invention, the rotor may be located next to, or on the side of the stator. According to some embodiments of the present invention, the rotor may be supported by ball bearings placed between the inner side of the stator and the exterior of the rotor. According to some embodiments of the present invention, the rotor may be supported by support wheels placed between the inner side of the stator and the exterior of the rotor. According to some embodiments of the present invention, the support wheels may be attached to the stator. According to some embodiments of the present invention, at least one of the edges of the rotor may be beveled. According to some embodiments of the present invention, the support wheels may be attached to the rotor. According to some embodiments of the present invention, at least one of the edges of the stator may be beveled. According to some embodiments of the present invention, at least one of the support wheels may be grooved or may be a train type of wheel. According to some embodiments of the present invention, a track or a guide may be attached to the stator or the rotor. According to some embodiments of the present invention, at least two carriages may be attached to the rotor or stator. According to some embodiments of the present invention, at least one carriage may be engaged with the track or guide. According to some embodiments of the present invention, the carriage may have at least one wheel attached to it. According to some embodiments of the present invention, the carriage may ride on the track or guide using at least one wheel.

According to some embodiments of the present invention, there may be at least two friction wheels attached to the stator which may clutch the rotor. According to some embodiments of the present invention, at least one of the friction wheels may be a support wheel. According to some embodiments of the present invention, the rotor may have teeth on, or substantially near its circumference. According to some embodiments of the present invention, there may be a C-shaped cogwheel attached to the rotor. According to some embodiments of the present invention, there may be at least two cogwheels attached to the stator, at least one of which may be engaged with the cogwheel attached to the rotor or the teeth on the rotor. According to some embodiments of the present invention, there may be a motor attached to the stator which may drive the cogwheels or the friction wheels attached to the stator. According to some embodiments of the present invention, the motor may be connected to the cogwheel directly or by a belt or by a chain or by a gear, or by a shaft, or in any other way. According to some embodiments of the present invention, the motor may turn the cogwheel or friction wheel, which in turn may rotate the rotor. According to some embodiments of the present invention, the rotor and the stator may be adapted to be the rotor and stator of a motor, and the rotor may be rotated by applying electrical current to the stator's coils.

According to some embodiments of the present invention, the X-ray source, and optionally the collimator, may be attached to the rotor. According to some embodiments of the present invention, the X-ray source may be attached to the inner side of the rotor opposite the rotor's open sector. According to some embodiments of the present invention, the source may be attached to the rotor, but may be physically located next, or side by side the rotor, and may face the rotor's rotation axis. According to some embodiments of the present invention, the detector may be attached to the rotor, opposite the X-ray source. According to some embodiments of the present invention, the detector may be attached to the inner side of the stator and may face the center of the stator. According to some embodiments of the present invention, the detector may be attached to the stator, but may be physically located next, or side by side to the stator, and may face the rotor's rotation axis. According to some embodiments of the present invention, the detector may be stationary and may span an angular range greater than 180°. According to some embodiments of the present invention, the controller may control the rotor's rotation. According to some embodiments of the present invention, the controller may control the actuator. According to some embodiments of the present invention, the controller may activate and control the radiation from the X-ray source. According to some embodiments of the present invention, the controller may acquire projection data from the detector. According to some embodiments of the present invention, the controller may reconstruct the projection data into images. According to some embodiments of the present invention, electrical and/or communication interface between the stator and the rotor may be provided. According to some embodiments of the present invention, electrical and/or communication interface between the controller and the rotor may be provided. According to some embodiments of the present invention, the electrical and/or communication interface may be by contact slip rings and brushes. According to some embodiments of the present invention, the electrical and/or communication interface may be by capacitive pickup interfacing. According to some embodiments of the present invention, the electrical and/or communication interface may be by inductive interfacing. According to some embodiments of the present invention, the electrical and/or communication interface may be by electrical cords connected between the stator and the rotor. According to some embodiments of the present invention, the gantry may be positioned vertically. According to some embodiments of the present invention, the gantry may be positioned horizontally. According to some embodiments of the present invention, the gantry may be positioned tilted.

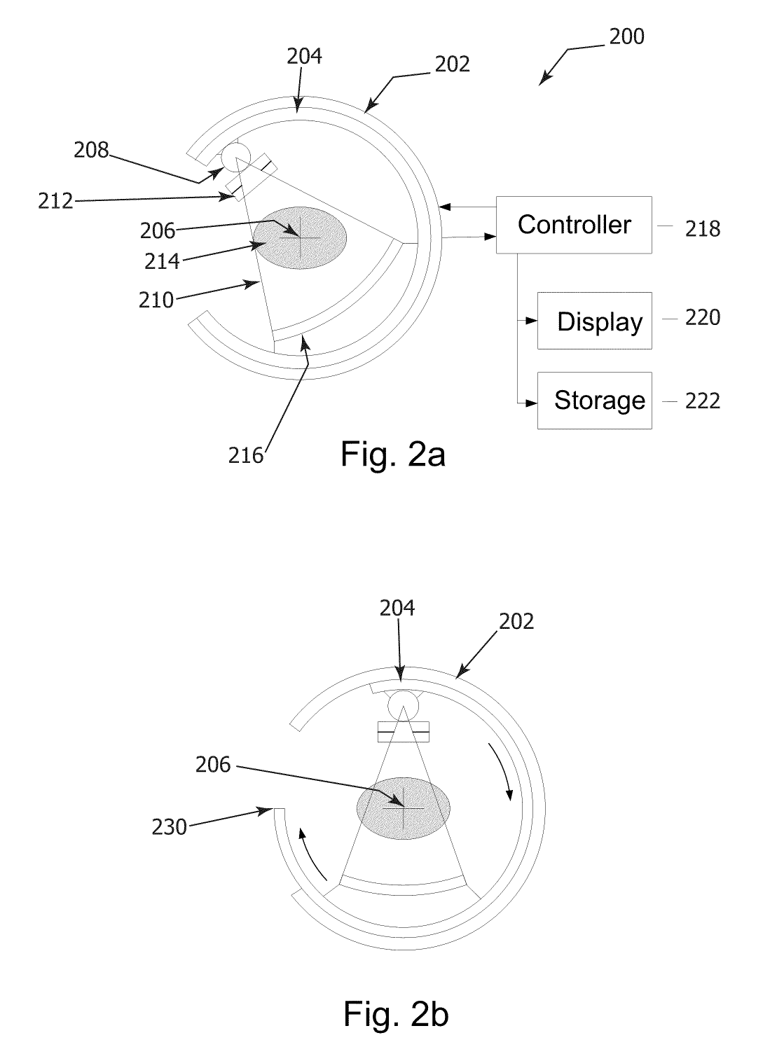

FIG. 2a is an exemplary schematic illustration of some embodiments according to the present invention. CT scanning system 200 may comprise a stationary gantry element 202, (stator) and a rotating gantry element 204, (rotor). Rotor 204 may be capable of rotating relative to stator 202 about rotation axis 206. X-ray source 208, may be mounted on rotor 204, and may emit X-ray beam 210 which may be collimated by collimator 212 and directed to scanned subject 214 and detector 216. In the example of FIG. 2a, detector 216 is shown to be mounted on rotor 204 opposite source 208 in a rotate-rotate third generation CT scanner configuration. Controller 218 may control the rotation of the rotor 204 about axis 206. The controller may also activate and control the intensity of X-ray source 208, and may acquire projection data from detector 216 and may also reconstruct the projection data to images. Subsystem 220 may display the images and subsystem 222 may store the images. The rotor and stator in system 200 may span across the (X,Y) plane, perpendicular to the rotation axis Z, in a similar manner to conventional “O” shaped CT scanners and unlike certain conventional C-arms used for CT scanning. Stator 202 and rotor 204 are exemplary schematic illustrations demonstrating a possible geometrical relation between them. According to some embodiments of the present invention, the stator and rotor may have a different outline and may have additional parts integrated to them, which are not shown here for clarity.

One aspect of the present invention is the open “C-shaped” structure of both the stator and the rotor. The “C shaped” structure may be a generally circular structure encompassing an interior area of more than 180° and less than 360°. FIG. 2a shows an example of the rotor positioned angularly in a way that the missing sectors in the rotor and stator overlap. In such a situation, the scanned subject 214 may be inserted into the scan field laterally through the missing sectors of the stator and rotor. By “inserting the subject into the scan field laterally” is meant inserting the subject from a direction radial to the gantry rotation axis. Each of the C shaped stator and rotor may cover over 180° of angular range around the center of rotation, therefore, at any rotation angle there may be some overlap between the stator and the rotor. According to some embodiments of the present invention, the stator or the rotor may cover an angular range of less than 180° around the center of rotation as long as both rotor and stator together cover more than 360° such that at any rotation angle there may be some overlap between the stator and the rotor.

The gantry according to some embodiments of the invention may be used vertically, horizontally or in other orientations.

Examples of situations in which the geometry according to some embodiments of the present invention is beneficial, are scans of patients lying on non-cantilever supports, walking patients that may walk into the scan field of a horizontal scanner, long industrial objects such as installed pipes, tree trunks, equine legs and other subjects in which it is more convenient to insert the subjects to the scan field through a side opening rather than through the scanner's bore.

FIG. 2b illustrates the gantry of system 200 with the rotor 204 rotated relative to stator 202 about the center of rotation 206. The rotor in the example of FIG. 2b is rotated approximately 40° clockwise (CW) relative to its position in FIG. 2a. At this rotation angle the “leading edge” 230 of the rotor may fill the missing sector of the stator.

FIGS. 2a and 2b show schematically the rotor 204 enclosed within stator 202 on the same (X,Y) plane. According to some embodiments of the present invention other geometries may also be possible. The stator and the rotor may be displaced in the Z direction (rotation axis direction) relative to each other. According to some embodiments of the present invention, the supporting part of the rotor may be enclosed within the stator, or the stator and the rotor may be mounted side by side along the rotation axis (the Z direction).

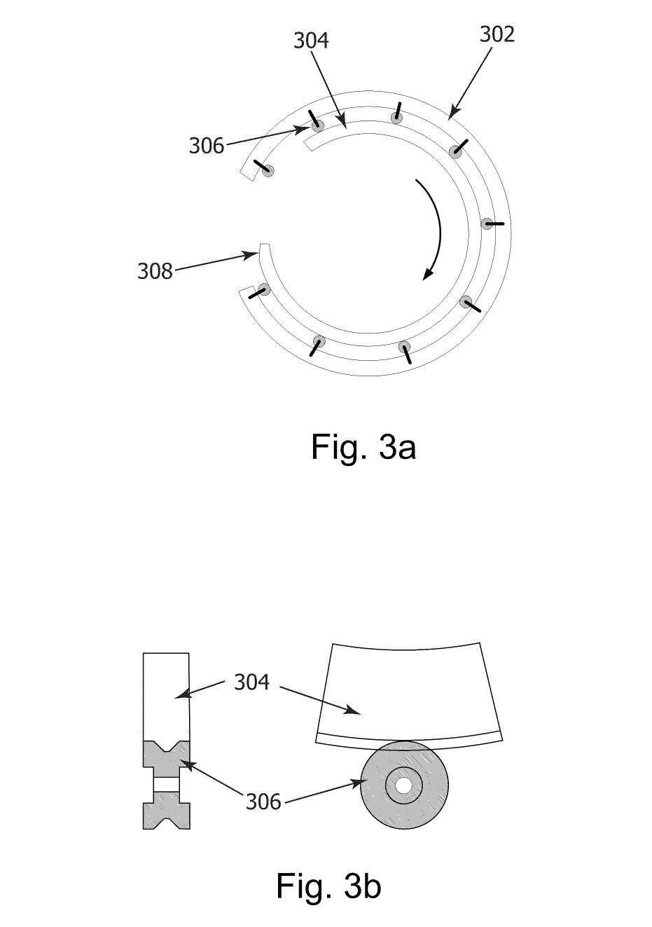

The mechanical interface between the stator and rotor may provide support to the rotor and may enable the rotation of the rotor relative to the stator about the center of rotation. FIG. 3a depicts a front view of an exemplary mounting system of the rotor to the stator according to some embodiments of the present invention. Subsystems such as the X-ray source, detector and other parts of the system are not shown in the figure so as not to obstruct its clarity. Stator 302 may support rotor 304 concentrically by multiple support wheels 306, enabling the rotation of rotor 304. Support wheels 306 may be spread over the angular range in order to provide support and centering of rotor 304 at any rotation angle. The leading edge 308 of rotor 304 may be beveled for smooth engagement with wheels 306 as the leading edge advances during rotation.

FIG. 3b is a further detailed illustration of the exemplary interface between the rotor 304 (a short sector is shown) and a support wheel 306 with a front and cross sectional views. In this example the support wheels 306 may be grooved and engaged with the rotor so as to provide lateral support in addition to the radial support.

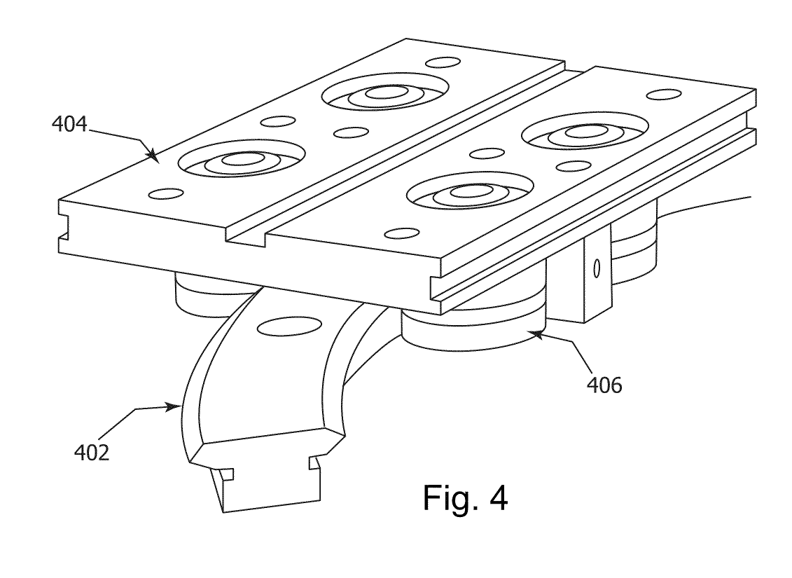

FIG. 4 is an illustration of an alternative exemplary arrangement for mechanical interfacing of the rotor and stator. A “C” shaped guide 402 may be mounted onto the stator (a section of the guide is shown). The rotor may be mounted onto multiple carriages 404 (one carriage shown). Carriages 404 may be engaged with guide 402 at certain rotation angles and may disengage from the guide over the missing sector. The carriages 404 may ride along the guide using wheels 406. Alternatively, a “C” shaped guide may be mounted on the rotor and multiple carriages may be mounted on the stator. Circular motion guides and matching carriages may be ordered from Bishop-Wisecarver Corporation of Pittsburg; CA, INA-Schaeffler KG of Homburg/Saar, Germany and other vendors.

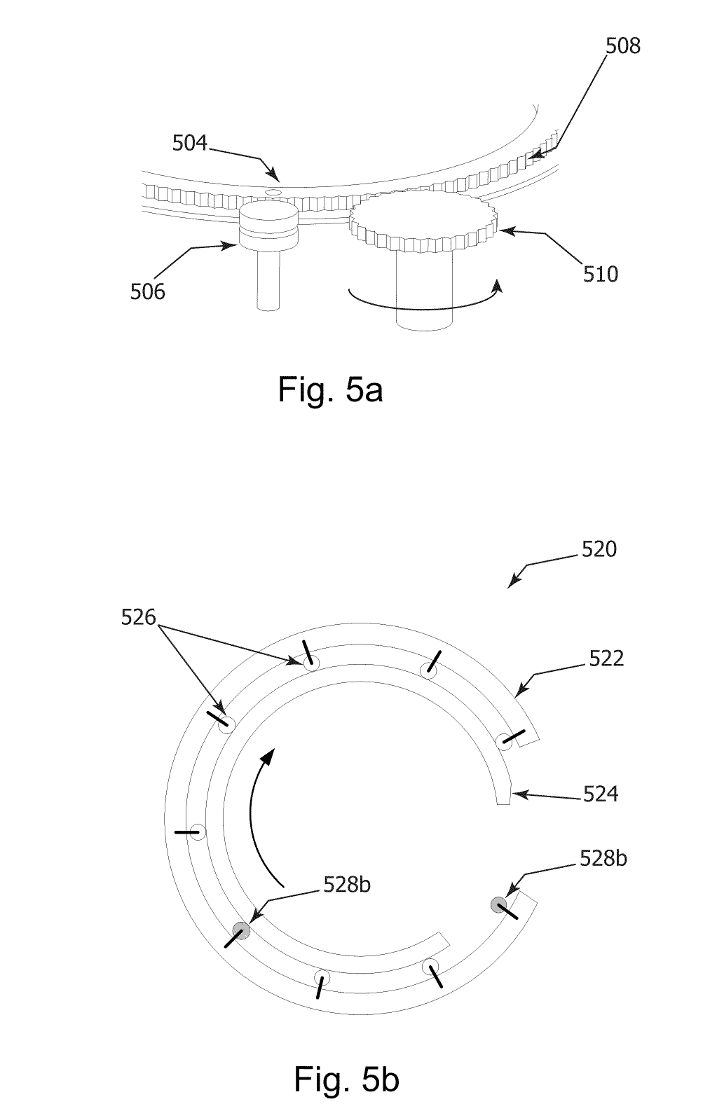

According to some embodiments of the present invention, the interface between the stator and rotor may provide means for driving the rotational motion of the rotor relative to the stator. According to some embodiments of the present invention, there may be means for driving the rotor relative to the stator. FIG. 5a is an illustration of an exemplary rotational drive mechanism according to some embodiments of the invention. The rotor 504 may be supported by support wheels 506 or by other means. A “C” shaped cogwheel 508 may be attached to the rotor concentric to the center of rotation. The rotor may be rotated by the driving cogwheels 510 which may be mounted on the stator. The driving cogwheels 510 may be coupled to a motor or motors (not shown) directly, via a gearbox, or by a belt or a chain, or in any other way. FIG. 5a shows only part of the rotor and one support wheel and drive cogwheel. Alternatively, according to some other embodiments of the present invention, the rotor may be supported by support wheels such as shown in exemplary FIGS. 3a and 3b. The rotor may use some or all of the support wheels also as drive wheels for rotating the rotor by friction.

FIG. 5b is an exemplary description of the system according to some other embodiments of the present invention. FIG. 5b depicts gantry 520 with stator 522 and rotor 524 which may be supported by multiple support wheels 526. Two wheels marked by numerals 528a and 528b may be interfaced to motors (not shown) and may be used to drive rotor 524. In the particular rotation angle shown in the example of FIG. 5b, wheel 528b is facing the missing sector of rotor 524 and only wheel 528a is engaged with the rotor and can drive the rotor. In some embodiments of the invention, more than two drive wheels may be provided.

According to some embodiments of the present invention, a direct drive motor for driving the rotor may be used. The direct drive motor may comprise an array of permanent magnets installed on the rotor and current coils installed on the stator, as may be known in the art.

FIGS. 3 to 5 are given only as examples. There may be other geometries and arrangements for interfacing the “C” shaped rotor to a “C” shaped stator and for revolving the rotor relative to the stator. All such other geometries and arrangements may also be within the scope of the present invention.

Electrical and communication interfaces between the rotor and the stator and/or the system controller may be required. Such interfaces may be used for transmission of power, control signals and data between the rotor and the stator and/or the controller. Various methods for rotor interfacing known in prior art CT may be applicable for the present invention, in some cases with obvious adaptations. For example, contact slip rings which may be used in some of the embodiments of the invention may require at least two sets of brushes separated angularly and operating in parallel so that when one brush is in front of the missing sector of the ring the other brush may perform the electrical contact. In a similar way, capacitive pickup contactless communication may require at least two sets of transmitters or receivers.

Other embodiments may comprise rotor electrical interfacing via cables which may be connected between the rotor and the stator. In these embodiments the rotor may not be able to rotate indefinitely, it may rotate in one direction for a limited angular range and then it may rotate in the opposite direction for a limited angular range. The interface cables may fold and unfold as the rotor rotates back and forth. Cable interface for CT scanners and the arrangements for cable folding and un-folding are well known in the art.

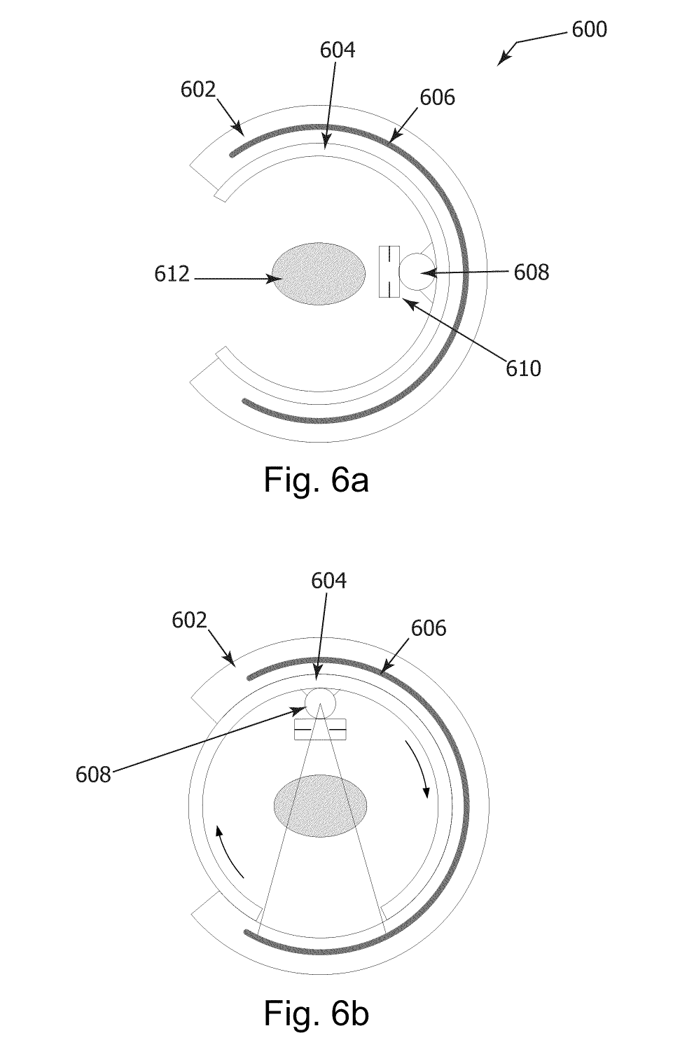

Reference is now made to FIG. 6a illustrating an exemplary fourth generation CT scanner 600 according to some embodiments of the invention. “C” shaped stator member 602 may carry rotating “C” shaped rotor member 604. Stationary detector 606 which may encompass an angular range larger than 180° around the center of rotation may also be mounted on the stator 602. Rotor 604 may carry X-ray source 608 and collimator 610 which may irradiate scanned subject 612. The example of FIG. 6a shows the system in the subject loading position wherein the missing sectors of the stator and rotor are substantially overlapping so as to allow subject positioning in the scan field through the opening.

FIG. 6b is an exemplary illustration of system 600 during scanning. Data acquisition may take place in the angular range of at least 180° while the source 608 is facing the active area of detector 606. The source and detector in FIGS. 6a and 6b may be shifted from the stator frame in the Z direction (parallel to the rotation axis) in a way that may enable the radiation from the source attenuated by the subject to impinge the detector with no interruption by the frame. In the example shown in FIGS. 6a and 6b the radiation may impinge the detector through the missing sector in the rotor. However, the source may be mounted in a different angular position on the rotor and radiation shadowing can still be avoided by shifting the source and detector from the rotor frame in the Z direction.

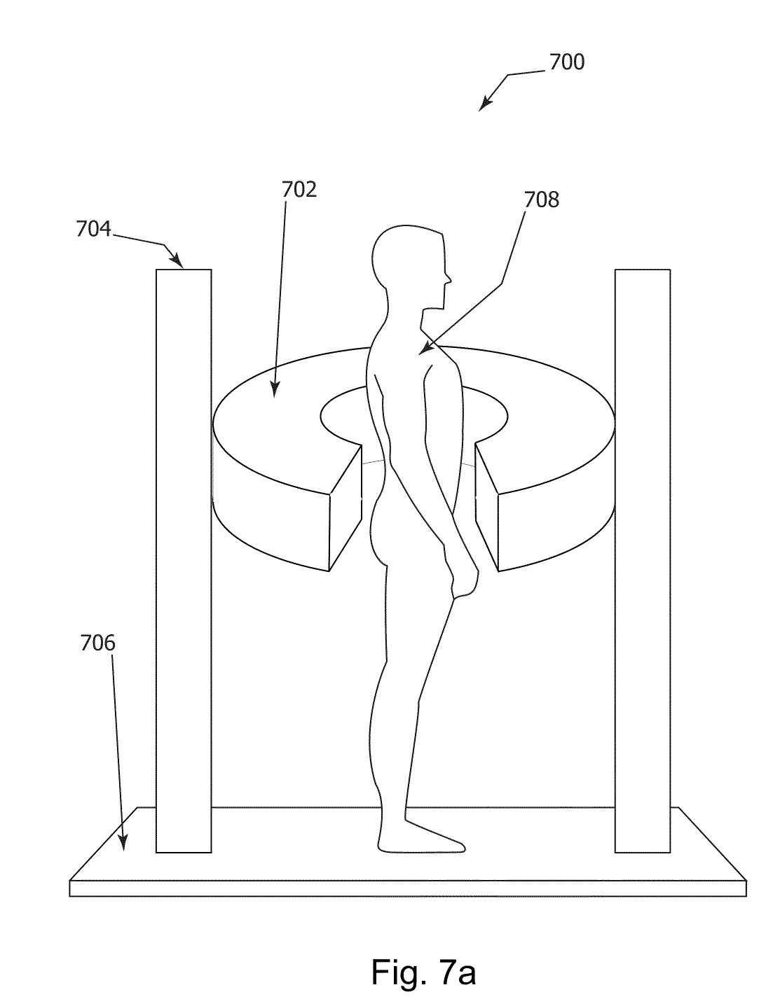

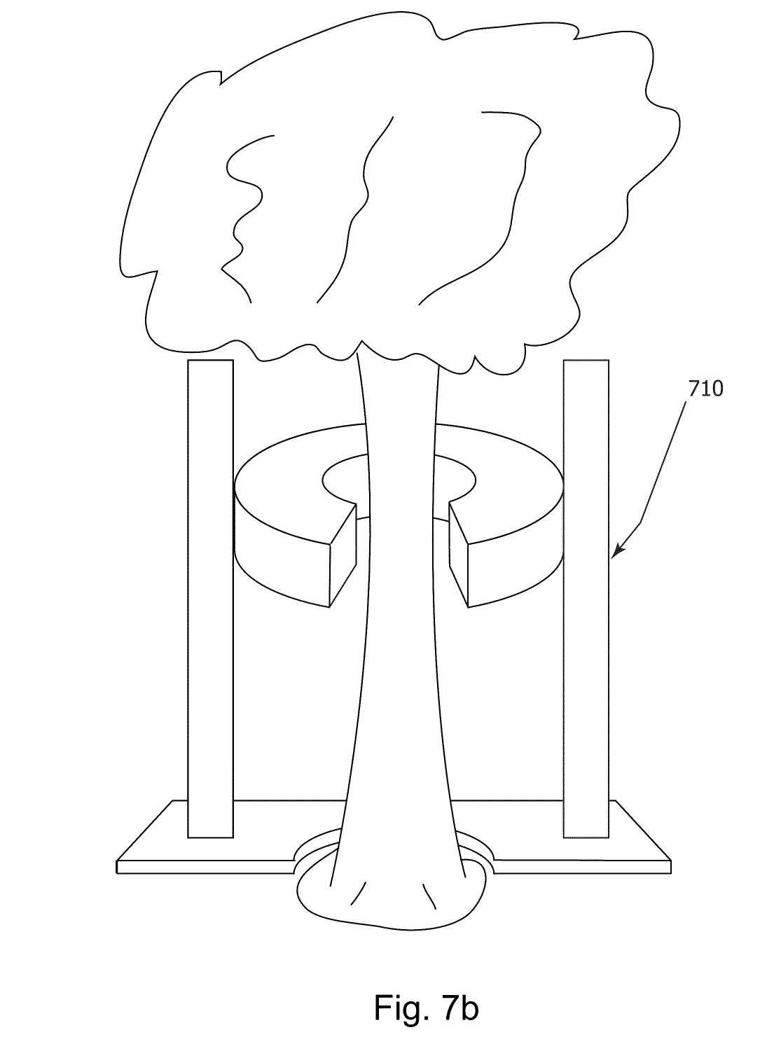

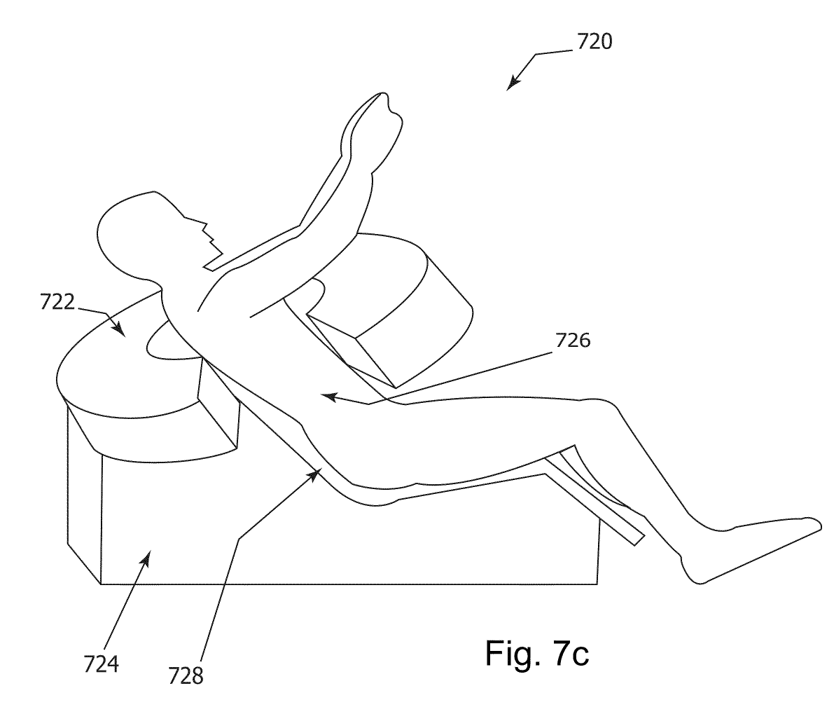

FIG. 7a illustrates an example of system 700 according to some embodiments of the invention. “C” shaped gantry 702 according the descriptions hereinabove may be supported horizontally by lift posts 704 over base 706. The gantry is shown in the loading position wherein the missing sectors of the stator and rotor overlap. A standing human patient 708 or another upright scanned subject may be positioned in the scan field through the gantry opening. Two posts 704 are shown in the drawing; however, other different geometries can be used, e.g. a single post with a cantilever supported gantry, more than two posts, or any other structure. A rectangular flat base is shown in the drawing; however other shapes and structures may be possible. Means may be provided in system 700 for lifting the gantry 702 relative to the base 706 during or between scans. Details of the lift are not provided as lifts suitable for this application are well known in various fields.

FIG. 7b illustrates system 710, similar in layout and functionality to system 700 of FIG. 7a, however adapted for use in forestry rather than in medicine. The base in system 710 has an opening so it may fit around a scanned subject, e.g. a trunk of a planted tree. Alternatively, system 710 may be cantilever supported on a stationary structure or a vehicle. Similar embodiments can be used to scan sections of long objects such as pipes, cables and the like wherein the scanner orientation is adapted to the orientation of the subject.

FIG. 7c illustrates an example of another system 720 according to some embodiments of the invention. “C” shaped gantry 722 may be in a tilted orientation and may be supported by base 724. The gantry is shown in an open side loading position. The base may also support seat 728 on which the patient may be sitting. System 720 may be useful for chest or heart scanning. Some of the advantages of system 720 over prior art scanners, such as shown for example in FIGS. 1a and 1b, may be smaller footprint and convenient positioning of the patient.

Exemplary systems 700, 710 and 720 shown in FIGS. 7a, 7b and 7c, respectively, are all shown in a loading position having the opening in the rotor aligned with the opening in the stator. According to some embodiments of the present invention, during scanning the rotor may fill the side opening in the gantry at least part of the time, as shown in the examples of FIGS. 2b and 6b.

Exemplary systems 700, 710 and 720 shown in FIGS. 7a, 7b and 7c, respectively, are provided only as examples, there may be other geometries and applications in which an apparatus according to embodiments of the present invention can be used. An apparatus according to embodiments of the present invention can be used in any orientation. For example, a horizontal gantry can be used for scanning a standing or upright sitting patient. A reclining patient supported by a dentist chair like support may be scanned by a tilted gantry. The scanner according to the present invention may be positioned on a floor, mobile on wheels, portable, handheld or have any mounting arrangement suitable for a particular application. In some cases the scanned subject may be brought to the scanner site and positioned in the scan field, in other cases the scanner may be brought to the subject site and may be mounted onto the subject for a scan. The size of the gantry can be made to fit particular body parts such as a whole body, head, limbs, etc., or it may fit a particular subject size for industrial and other applications.

Embodiments of the invention were described generally with reference to rotation of the source in a single plane relative to the scanned subject. Multiplicity of axial slices or segments may be scanned by moving the gantry relative to the subject in the axial direction in steps, either by moving the gantry parallel to the rotation axis or by moving the subject parallel to the rotation axis. According to some embodiments of the present invention, scanning may also involve movement of the source relative to the subject in the axial direction during scan, either by moving the source parallel to the rotation axis or by moving the subject parallel to the rotation axis, to form a helical scan as known in the art. According to some embodiments of the present invention, scanning may be done at any angular range around the subject, it may be 180°, less than 180°, more than 180°, 360°, or a higher angular range per scan. According to some embodiments of the present invention, the rotor rotation during scan may be counter clockwise, clockwise, or in alternating directions. According to some embodiments of the present invention, the rotor rotation may be at an approximately constant speed. According to some embodiments of the present invention, the rotor rotation may be at a variable speed.

The invention was generally described with reference to human medical scanning. However, the invention may also be applicable to other fields. “C” shaped CT according to the invention can be used to scan equine legs, giraffe's necks, standing trees, telephone poles, long pipes and other objects. In some of these examples there may be a benefit to a scanning device that can be mounted onto the scanned subject without moving or distracting the subject.

For clarity of description, the invention was described with a single focal spot X-ray as the emission source, however, the invention may be applicable also for X-ray sources comprising multiple focal spots emitting X-rays simultaneously or sequentially, or in any other emission mode. It may also be applicable to X-ray sources which may be made to move relative to the rotor during scan.

According to some embodiments of the present invention, the X-ray detectors may be any type of X-ray detectors known today or which may be devised in the future, suitable for CT scanning, for example, one and two dimensional arrays of scintillator-photodiode detector elements, arrays of direct conversion detector elements, gas detectors and flat panel detectors of various types. According to some embodiments of the present invention, the detectors may be used in any acquisition mode known today or which may be devised in the future, such as current integration mode, single photon counting mode or other modes.

The present invention was explained with reference to a “C shaped” stator and rotor. “C shaped” may refer to a generally circular frame encompassing the scanned subject by more than 180° and less than 360° relative to the rotation plain of the X-ray source, in a way that the subject may not be totally surrounded by the stator or the rotor. According to some embodiments of the present invention, the actual outline of the stator or rotor may not necessarily be “C shaped”. For example, the stator may have a frame of any suitable shape for supporting the scanner on the floor. Experts in the art will appreciate that embodiments of the present invention may be provided with external covers to protect the scanned subject and bystanders from rotating parts. Such covers may be adapted to expose side opening in the gantry for lateral subject loading and cover the side opening during rotational motion.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

What is claimed:

1. A Computer Tomography (CT) gantry for scanning a subject comprising:
a first semicircular stationary support element and a second semicircular rotational support element adapted to rotate concentric to said first support element by at least 360 degrees; and
wherein each of said first and second semicircular support elements has a missing sector and wherein said missing sectors are missing also during rotation.

2. The gantry according to claim 1, further comprising an X-ray source structurally associated with said rotating support element.

3. The gantry according to claim 1, further comprising an X-ray detector array structurally associated with said rotating support element.

4. The gantry according to claim 2, further comprising an electromechanical actuator adapted to rotate said rotating support element concentrically around a common axis of the two concentric support elements.

5. The gantry according to claim 4, wherein said subject is loaded to scan position in a lateral direction respective the gantry and wherein during subject loading said electromechanical actuator is adapted to rotate said rotating support element into a position such that the missing sectors of both support element are at least partially overlapping.

6. The gantry according to claim 4, wherein during subject scanning said electromechanical actuator is adapted to rotate said rotating support element at least 180 degrees relative to said stationary support element.

7. The gantry according to claim 4, wherein during subject scanning said electromechanical actuator is adapted to rotate said rotating support element at least 360 degrees relative to said stationary support element.

8. A Computer Tomography (CT) system for scanning a subject comprising:
a gantry comprising a first semicircular stationary support element and a second semicircular rotational support element adapted to rotate concentric to said first support element by at least 360 degrees; and
wherein each of said first and second semicircular support elements has a missing sector and wherein said missing sectors are missing also during rotation.

9. The system according to claim 8, further comprising an X ray source mounted on said rotational support element and adapted to irradiate said subject from multiple view angles.

10. The system according to claim 9, further comprising an X Ray detector array adapted to receive X ray radiation that was emitted by said source and attenuated by said subject.

11. The system according to claim 10, further comprising a control circuitry adapted to regulate: (1) relative rotational positions between said first and second elements, (2) activation of said X ray source, and (3) acquisition of attenuation data and reconstruction of CT images.

12. The system according to claim 11, wherein said control circuitry comprises a single controller.

13. The system according to claim 11, wherein said control circuitry comprises a multiplicity of controllers.

14. The system according to claim 11, further comprising an electromechanical actuator functionally associated with said control circuitry, wherein responsive to signaling from said control circuitry said actuator is adapted to rotate said rotating support element concentrically around a common center of the two concentric support elements.

15. The system according to claim 14, wherein said subject is loaded to scan position in a lateral direction respective the gantry and wherein during subject loading said control circuitry is adapted to cause said electromechanical actuator to rotate said rotating support element into a position such that the missing sectors of both support elements are at least partially overlapping.

16. The system according to claim 14, wherein during subject scanning said control circuitry is adapted to cause said electromechanical actuator to rotate said rotating support element at least 180 degrees relative to said stationary support element.

17. The system according to claim 16, wherein during scanning the subject is scanned during rotation of at least 180 degrees.

18. The system according to claim 16, wherein during scanning the subject is scanned during rotation of at least 360 degrees.

19. The system according to claim 8, wherein said gantry rotation plan is vertical and the scan subject is a human in a horizontal laying position.

20. The system according to claim 8, wherein said gantry rotation plan is horizontal and the scan subject is a human in a standing or upright sitting position.

21. The system according to claim 8, wherein the scanned subject is a human in reclining position.

22. The system according to claim 8, wherein the scanned subject is a long object loaded into the scan field in a lateral direction respective the gantry.

23. The system according to claim 8, wherein said gantry is pivotally movable and wherein the gantry is adapted to be moved and positioned around the subject without moving the subject.

24. A method of for performing Computer Tomography (CT) scanning of a subject comprising:
adjusting elements of a gantry having a first semicircular stationary support element and a second semicircular rotational support element adapted to rotate concentric to said first support element by at least 360 degrees such that missing sectors of each of the support elements at least partially overlap and form an opening between the inside and outside of the gantry; and
loading a scan subject into a scan area within the gantry laterally respective the gantry rotation axis.

25. The method according to claim 24, further comprising activating an X ray source mounted on said second support element to irradiate the subject from multiple view angles.

26. The method according to claim 25, further comprising acquiring data from an X Ray detector array adapted to receive X ray radiation that was emitted by the source and attenuated by the subject.

27. The method according to claim 26, further comprising activating control circuitry to regulate: (1) relative rotational positions between said first and second elements, (2) activation of said X ray source, and (3) acquisition of attenuation data and reconstruction of CT images.

28. The method according to claim 27, further comprising actuating an electromechanical actuator responsive to signaling from the control circuitry to rotate the rotating support element concentrically around a common center of the two concentric support elements.

29. The method according to claim 27, wherein during subject loading cause the electromechanical actuator to rotate the rotating support element into a position such that the missing sectors of both support elements are at least partially overlapping.

30. The method according to claim 27, wherein during subject scanning causing the electromechanical actuator to rotate the rotating support element at least 180 degrees relative to said stationary support element.

31. The method according to claim 30, wherein during scanning the subject is scanned during rotation of at least 180 degrees.

32. The method according to claim 30, wherein during scanning the subject is scanned during rotation of at least 360 degrees.

33. The method according to claim 24, wherein the gantry rotation plan is vertical and the scan subject is a human in a horizontal laying position.

34. The method according to claim 24, wherein the gantry rotation plan is horizontal and the scan subject is a human in a standing or upright sitting position.

35. The method according to claim 24, wherein the scanned subject is a human in reclining position.

36. The method according to claim 24, wherein the scanned subject is a long object loaded into the scan field in a lateral direction respective the gantry.

37. The method according to claim 24, further comprising moving the gantry around the subject without moving the subject.

38. The method according to claim 24, wherein multiplicity of axial segments of the scanned subject are scanned sequentially by changing the axial position of the gantry relative to the subject in steps.

39. The method according to claim 24, wherein multiplicity of axial segments of the scanned subject are scanned helically by changing the axial position of the gantry relative to the subject during scan.
